(19) United States
(12) Patent Application Publication
(10) Pub. No.: US 2014/0056865 A1
(43) Pub. Date: Feb. 27, 2014

(54) ADIPOSE COMPOSITION SYSTEMS AND METHODS

(71) Applicant: AlloSource, Centennial, CO (US)

(72) Inventors: Adrian C. Samaniego, Parker, CO (US); Derek J. Miller, Denver, CO (US)

(73) Assignee: AlloSource, Centennial, CO (US)

(21) Appl. No.: 13/970,324

(22) Filed: Aug. 19, 2013

Related U.S. Application Data
(60) Provisional application No. 61/684,386, filed on Aug. 17, 2012.
(60) Provisional application No. 61/715,969, filed on Oct. 19, 2012.
(60) Provisional application No. 61/716,009, filed on Oct. 19, 2012.
(60) Provisional application No. 61/775,200, filed on Mar. 8, 2013.

(51) Int. Cl. A61K 47/46; A61K 35/32; A61K 35/28

(52) U.S. Cl. CPC A61K 47/46; A61K 35/28; A61K 35/32
USPC 424/93.72; 514/773; 424/93.7; 424/549; 514/1.1

(57) ABSTRACT

Embodiments of the present invention encompass compositions containing an adipose derived carrier, matrix, or filler, in some cases optionally in combination with bone particles or other granular materials or substances, for delivery to a human patient. Methods of manufacture and use of such adipose derived compositions are also disclosed.

### CROSS-REFERENCES TO RELATED APPLICATIONS

This application is a nonprovisional of, and claims the benefit of the filing date of, U.S. Provisional Patent Application Nos. 61/684,386 filed Aug. 17, 2012, 61/715,969 and 61/716,009 both filed Oct. 19, 2012, and 61/775,200 filed Mar. 8, 2013. The entire content of each of the above filings is incorporated herein by reference.

### BACKGROUND OF THE INVENTION

Embodiments of the present invention relate generally to medically useful compositions, and in particular to adipose derived filler materials, matrix systems, carrier systems, and allogeneic medical graft compositions, and methods of their use and manufacture.

Human tissue compositions, which may be derived from cadaveric donors, have been used for many years in various surgical procedures, including treatments for certain medical conditions, including tissue defects and wounds and in reconstructive surgical procedures.

Medical grafting procedures often involve the implantation of autogenous, allograft, or synthetic grafts into a patient to treat a particular condition or disease. The use of musculoskeletal allograft tissue in reconstructive orthopedic procedures and other medical procedures has markedly increased in recent years, and millions of musculoskeletal allografts have been safely transplanted. A common allograft is bone. Typically, bone grafts are reabsorbed and replaced with the patient's natural bone upon healing. Bone grafts can be used in a variety of indications, including neurosurgical and orthopedic spine procedures for example. In some instances, bone grafts can be used to fuse joints or to repair broken bones. In some cases, bone material is combined with mesenchymal stem cells to produce a graft composite.

Allograft and autogenous tissue are both derived from humans; the difference is that allograft is harvested from an individual (e.g. donor) other than the one (e.g. patient) receiving the graft. Allograft tissue is often taken from cadavers that have donated their tissue so that it can be used for living people who are in need of it, for example, patients whose bones have degenerated from cancer. Such tissues represent a gift from the donor or the donor family to enhance the quality of life for other people.

Medically useful tissues may also have reconstructive applications. For example, currently known reconstructive techniques are used to fill a lumpectomy often using either the patient's own fat from a secondary surgical site. In such cases, healing of the secondary surgical site may result in a depression or divot. Relatedly, in some cases, foreign implantable material is used to fill a lumpectomy, however such techniques may result in rejection (e.g. the material becomes removed from the body) or encapsulation (e.g. the material creates an unnatural shape or lump).

Hence, although presently used reconstructive surgical techniques and tissue graft compositions and methods provide real benefits to patients in need thereof, still further improvements are desirable. Embodiments of the present invention provide solutions to at least some of these outstanding needs.

### BRIEF SUMMARY OF THE INVENTION

Adipose derived carrier systems and methods can be used to deliver various types of particles to a treatment site within the human body. For example, an ostebiologic composition containing bone particles combined with an adipose derived carrier can be administered to a patient.

In one aspect, embodiments of the present invention encompass methods of manufacturing an allogeneic adipose derived carrier for implantation into a patient. Exemplary methods include decellularizing an amount of adipose tissue, separating a stromal vascular fraction (SVF) from the decellularized adipose tissue, and extracting an organic phase from the decellularized adipose tissue following the SVF separation. In some cases, methods include combining the organic phase with a granular tissue material. In some cases, the granular tissue material includes bone particles. Optionally, the granular tissue material may include cortical bone particles, cancellous bone particles, or both. In some cases, the granular tissue material includes bone particles and stem cells. The decellularizing step may include treating the amount of adipose tissue with collagenase. According to some embodiments, the organic phase extraction step includes treating the decellularized adipose tissue with a base solution, an alkaline alcohol solution, or an alkaline organic solution. In some instances, the base, alkaline alcohol, or alkaline organic solution includes sodium hydroxide. Embodiments of the present invention further encompass techniques that involve combining the organic phase with a granular tissue material, where the adipose tissue and the granular tissue material are from a common donor individual.

In another aspect, embodiments of the present invention encompass adipose derived carrier compositions for use in medical treatment procedures or surgeries. An exemplary allogeneic adipose derived carrier, for implantation into or administration to a patient, may include an organic phase of decellularized adipose tissue that is substantially free of a stromal vascular fraction. In some instances, the carrier composition also includes a granular tissue material. For example, the granular tissue material may include bone particles. In some instances, the granular tissue material may include cortical bone particles, cancellous bone particles, or both. In some instances, the granular tissue material includes bone particles and stem cells. According to some embodiments, the organic phase of decellularized adipose material and the granular tissue material are from a common donor individual.

In still another aspect, embodiments of the present invention encompass methods of delivering a granular tissue material to a patient. Exemplary methods may involve administering the granular tissue material combined with a carrier to a treatment site of the patient, where the carrier includes an organic phase of decellularized adipose tissue that is substantially free of a stromal vascular fraction. In some instances, the granular tissue material includes bone particles. In some instance, the granular tissue material includes cortical bone particles, cancellous bone particles, or both. In some instances, the granular tissue material includes bone particles and stem cells. Optionally, the organic phase of decellularized adipose tissue and the granular tissue material may be recovered from a common donor individual.

In another aspect, embodiments of the present invention encompass methods of manufacturing an allogeneic adipose derived carrier for implantation into a patient. Exemplary methods include obtaining a decellularized adipose tissue that has been recovered from a human donor, where an amount of stromal vascular fraction has been separated from the decellularized adipose tissue. Methods may also include exposing the decellularized adipose tissue to an alkaline organic solution so as to extract an organic phase, and processing the organic phase to obtain a carrier. According to some embodiments, the carrier includes randomly oriented single chain collagen polypeptide fragments from an extracellular matrix of the decellularized adipose tissue. In some cases, methods may further include combining the carrier with a granular tissue material. In some cases, the granular tissue material may include bone particles. In some cases, the granular tissue material may include cortical bone particles, cancellous bone particles, or both. In some cases, the granular tissue material may include bone particles, optionally along with stem cells. According to some embodiments, the processing protocol includes centrifuging the organic phase. In some cases, the obtained decellularized adipose tissue has been treated with collagenase. In some cases, the alkaline organic solution includes sodium hydroxide, ethanol, methanol, isopropanol, benzene, potassium hydroxide, or calcium hydroxide, or any mixture or combination thereof. In some cases, methods further include combining the carrier with a granular tissue material, where the decellularized adipose tissue and the granular tissue material are from the same donor individual (e.g. tissue materials obtained from a common donor).

In another aspect, embodiments of the present invention encompass an allogeneic adipose derived carrier for implantation into a patient. Exemplary carriers may include an organic phase of decellularized adipose tissue from a human donor that has been exposed to an alkaline organic solution to produce randomly oriented single chain collagen polypeptide fragments from an extracellular matrix of the decellularized adipose tissue. In some cases, the organic phase or carrier is substantially free of a stromal vascular fraction. In some cases, the adipose carrier is combined with a granular a granular tissue material. In some cases, the granular tissue material can include bone particles. In some cases, the granular tissue material can include a cortical bone particle component (e.g. one or more cortical bone particles), and a cancellous bone particle component (e.g. one or more cancellous bone particles), or both. According to some embodiments, the granular tissue material includes bone particles and stem cells. According to some embodiments, the organic phase of decellularized adipose material and the granular tissue material are derived or obtained from the same donor individual.

In still another aspect, embodiments of the present invention encompass methods of delivering a substance to a patient. Exemplary methods may include administering the substance combined with a carrier to a treatment site of the patient. In some cases, the carrier is derived from a human donor and includes an organic phase of decellularized adipose tissue that has been exposed to alkaline organic solution to produce randomly oriented single chain collagen polypeptide fragments from an extracellular matrix of the decellularized adipose tissue and is substantially free of a stromal vascular fraction. In some cases, the substance includes granular tissue material. In some cases, the granular tissue material includes bone particles. In some cases, the granular tissue material includes cortical bone particles, cancellous bone particles, or both. In some cases, the granular tissue material includes bone particles and stem cells.

Adipose derived matrix systems and methods can be used to deliver various types of materials to a treatment site within the human body. For example, an ostebiologic composition containing cells, proteins, and/or large molecules, combined with an adipose derived matrix, can be administered to a patient.

In one aspect, embodiments of the present invention encompass methods of manufacturing an allogeneic adipose derived matrix for implantation into a patient. Exemplary methods include decellularizing an amount of adipose tissue, separating a stromal vascular fraction (SVF) from the decellularized adipose tissue, extracting an organic phase from the decellularized adipose tissue following the SVF separation, and processing the organic phase to provide the adipose derived matrix. In some cases, manufacturing methods may include combining the matrix with cells, proteins, and/or other large molecules. In some cases, the decellularizing step may include treating the amount of adipose tissue with collagenase. In some cases, the organic phase extraction step may include treating the decellularized adipose tissue with a base solution. In some cases, the base solution may include sodium hydroxide.

In another aspect, embodiments of the present invention encompass an allogeneic adipose derived matrix for implantation into a patient. The matrix may include a processed organic phase of decellularized adipose tissue that is substantially free of a stromal vascular fraction. In some cases, the matrix may also include or be combined with cells, proteins, and/or other large molecules. In some cases, the processed organic phase of decellularized adipose material and the cells, proteins, and/or other large molecules are from a common donor individual.

In another aspect, embodiments of the present invention encompass methods of delivering a treatment material to a patient. Exemplary methods may include administering the treatment material combined with a matrix to a treatment site of the patient. The matrix may include a processed organic phase of decellularized adipose tissue that is substantially free of a stromal vascular fraction. In some cases, the treatment material includes mesenchymal stem cells or platelet-rich plasma. In some cases, the treatment material includes cells, proteins, and/or other large molecules. In some cases, the processed organic phase of decellularized adipose tissue and the treatment material are from a common donor individual.

In another aspect, embodiments of the present invention encompass methods of manufacturing an allogeneic adipose derived matrix for implantation into a patient. Exemplary methods may include obtaining a decellularized adipose tissue that has been recovered from a human donor, where an amount of stromal vascular fraction has been separated from the decellularized adipose tissue. Further, methods may include exposing the decellularized adipose tissue to alkaline organic solution to extract an organic phase, processing the organic phase, and exposing the processed organic phase to a polar solution to remove oils and aqueous content to provide an adipose derived matrix. The adipose derived matrix may include randomly oriented single chain collagen polypeptide fragments from an extracellular matrix derived from the decellularized adipose tissue. In some cases, methods further include combining the matrix with cells, proteins, other large molecules, or any combination thereof. In some cases, the adipose tissue, the cells, the proteins, and/or the large molecules are from the same donor individual. In some cases, the polar solution includes 1-propanol, ethanol, acetone, and/or methanol. According to some embodiments, methods may further include combining the adipose derived matrix material with mesenchymal stem cells, platelet-rich plasma, or both. According to some embodiments, the decellularized adipose tissue has been treated with collagenase. According to some embodiments, the basic solution includes sodium hydroxide, ethanol, methanol, isopropanol, benzene, potassium hydroxide, and/or calcium hydroxide. According to some embodiments, methods include obtaining mesenchymal stem cells from the amount of stromal vascular fraction.

In another aspect, embodiments of the present invention encompass methods of delivering a treatment material to a patient. Exemplary methods may include administering the treatment material combined with an adipose derived matrix to a treatment site of the patient. The adipose derived matrix may include an organic phase of decellularized adipose tissue that has been obtained from a human donor and exposed to alkaline organic solution to produce randomly oriented collagen fibers from an extracellular matrix derived from adipose tissue, that is substantially free of a stromal vascular fraction, and that has been exposed to a polar solution to remove oils and aqueous content. According to some embodiments, the treatment material comprises includes cells, proteins, and/or other large molecules. According to some embodiments, the polar solution includes 1-propanol, ethanol, and/or methanol. According to some embodiments, the treatment material includes mesenchymal stem cells and/or platelet-rich plasma. According to some embodiments, the organic phase of decellularized adipose tissue and the treatment material are from a common (i.e. the same) donor individual.

In another aspect, embodiments of the present invention encompass an allogeneic adipose derived matrix for implantation into a patient. Exemplary adipose derived matrix materials may include an organic phase of decellularized adipose tissue that has been obtained from a human donor and exposed to alkaline organic solution to produce randomly oriented collagen fibers from an extracellular matrix derived from adipose tissue, that is substantially free of a stromal vascular fraction, and that has been exposed to a polar solution to remove oils and aqueous content. In some cases, the adipose derived matrix may include cells, proteins, and/or other large molecules. According to some embodiments, the organic phase of decellularized adipose material and the cells, proteins, and/or other large molecules are from a common donor individual. In some cases, the adipose derived matrix is substantially free of oils or lipid. In some cases, the adipose derived matrix is in a dry powder form. In some cases, the adipose derived matrix can be combined with a concentrated mesenchymal stem cell slurry or a platelet-rich plasma slurry. Optionally, the adipose matrix material can be cryopreserved. In some cases, the polar solution includes 1-propanol, ethanol, and/or methanol.

Embodiments of the present invention encompass tissue graft compositions which include mesenchymal (adult) stem cells combined with partially demineralized bone and an adipose carrier or matrix. Exemplary compositions may be prepared as a live cell bone graft substitute, for example.

Tissue graft compositions as disclosed herein are well suited for use as a substitute for autograft compositions, and thus can eliminate the need for an autograft patient recover site, thereby avoiding potential morbidity and pain. Further, exemplary compositions can provide a biologic solution for fusion applications (e.g. spinal fusions), and can present osteoconductive, osteoinductive, and/or osteogenic potential. In some cases, compositions can be delivered to patients presenting with bone fractures or defects, optionally as non-union breaks, including rib, spine, joint, and periodontal bones. Optionally, the graft material may be applied via a cage mechanism. Tissue graft compositions can be prepared as a stem cell graft material that is lyophilized or freeze dried. For example, adipose derived stem cell material can be seeded on or combined with a demineralized bone material, and cryopreserved for later use, storage, or transport. In use, the mesenchymal stem cells can adhere or bond to the bone substrate.

According to some embodiments, tissue compositions can be prepared as a live cellular bone growth substitute, such as an adult stem cell graft. In some cases, adult stem cells are recovered from adult human organ and tissue donors. Exemplary stem cell bone growth substitutes or adult stem cell bone graft materials can be recovered from adult human adipose tissue and is processed and cryopreserved into a stem cell bone graft for use by surgeons to promote bone growth and healing. In some cases, donated human (allograft) bone is recovered and subjected to a demineralization process. In some cases, donaged human (allograft) adipose tissue is recovered, optionally from the same donor from which the bone is recovered, and processed to collect cells and other materials present in the adipose tissue.

Exemplary tissue graft materials can be prepared as a putty or gel, and can be provided to a user in a ready to use packaged formulation which does not require rinsing before administration to a patient.

In one aspect, embodiments of the present invention encompass composite allograft materials prepared from tissue obtained from an individual human donor. Exemplary composite graft materials include a stem cell component and a bone component. Optionally, the composite graft material may include an adipose component.

In another aspect, embodiments of the present invention encompass composite allograft materials prepared from tissue obtained from an individual human donor. Exemplary composite materials or compositions include a mesenchymal stem cell component, a bone component, and an adipose component. In some cases, the adipose component includes an organic phase of decellularized adipose tissue that has been exposed to alkaline organic solution and that is substantially free of a stromal vascular fraction. In some cases, the bone component is at least partially demineralized. In some cases, the bone component includes cancellous bone. In some cases, the adipose component is lyophilized. In some cases, the adipose component includes an adipose carrier. In some cases, the allograft material presents osteoconductive, osteoinductive, or osteogenic potential. or a combination thereof.

In still another aspect, embodiments of the present invention encompass methods of manufacturing a composite allograft material for implantation into a patient. Exemplary methods may include obtaining adipose tissue that has been recovered from a human donor, obtaining bone tissue that has been recovered from the human donor, combining the bone tissue with stem cells, and combining the stem cells and the bone tissue with an adipose carrier obtained from the adipose material. The adipose carrier may include an organic phase of decellularized adipose tissue that has been exposed to alkaline organic solution and that is substantially free of a stromal vascular fraction. In some cases, methods may include exposing the adipose material to an enzymatic digestion material. In some cases, methods may include exposing the combined stem cells and bone tissue to a cryopreservative. In some cases, the cryopreservative may include dimethyl sulfoxide. In some cases, methods may include exposing the combined stem cells and bone tissue to the cryopreservative for about 5 seconds or less. In some cases, methods may include subjecting the bone tissue to a demineralization process. In some cases, methods may include extracting the stem cells from the adipose material. The stem cells may include mesenchymal stem cells. In some cases, methods may include decellularizing an amount of stem cell free adipose tissue. In some cases, methods may include neutralizing the amount of stem cell free adipose tissue.

In yet another aspect, embodiments of the present invention encompass methods of delivering a treatment material to a patient. Exemplary methods may include administering the treatment material to the patient, where the treatment material includes a stem cell component, a bone component, and an adipose component. The bone component may be at least partially demineralized, and the adipose component may include an organic phase of decellularized adipose tissue that has been derived from a human donor and exposed to alkaline organic solution and that is substantially free of a stromal vascular fraction. In some cases, the adipose component includes an adipose derived carrier. In some cases, the stem cell component includes mesenchymal stem cells. In some cases, the treatment material is present as a gel or a putty.

In another aspect, embodiments of the present invention encompass composite allograft materials prepared from tissue obtained from an individual human donor. Exemplary composite allograft materials or compositions include a stem cell component, a bone component, and an adipose carrier. In some cases, the adipose derived carrier may include a processed organic phase of decellularized adipose tissue that has been exposed to alkaline organic solution and that is substantially free of a stromal vascular fraction. In some cases, the stem cell component includes mesenchymal stem cells. In some cases, the bone component includes cortical bone particles, cancellous bone particles, or both. In some cases, the bone component and the stem cell component are treated with a cryopreservative. In some cases, the bone component is at least partially demineralized. In some cases, the decellularized adipose tissue has been exposed to collagenase. In some cases, the organic phase has been centrifuged to separate the adipose carrier from excess water. In some cases, the basic solution includes sodium hydroxide, ethanol, methanol, isopropanol, benzene, potassium hydroxide, and/or calcium hydroxide. In some cases, the adipose carrier is lyophilized.

In still yet another aspect, embodiments of the present invention encompass methods of manufacturing a composite allograft material for implantation into a patient. Exemplary methods may include obtaining adipose tissue that has been recovered from a human donor, separating a stromal vascular fraction from the adipose tissue, exposing the adipose tissue to alkaline organic solution to extract an organic phase, processing the organic phase to obtain a carrier, obtaining bone tissue that has been recovered from the human donor, combining the bone tissue with stem cells, and combining the stem cells and the bone tissue with the carrier. In some cases, methods may include exposing the adipose tissue to an enzymatic digestion material. In some cases, methods may include treating the combined stem cells and bone tissue with a cryopreservative, for example by briefly exposing the stem cells and bone tissue to a cryopreservative. In some cases, methods may include subjecting the bone tissue to a demineralization process. In some cases, methods may include extracting the stem cells from adipose tissue of the individual donor. In some cases, the basic solution may include sodium hydroxide, ethanol, methanol, isopropanol, benzene, potassium hydroxide, and/or calcium hydroxide. In some cases, the carrier is lyophilized.

In another aspect, embodiments of the present invention encompass methods of delivering a treatment material to a patient. Exemplary methods may include administering the treatment material to the patient, where the treatment material includes a mesenchymal stem cell component, a bone component, and an adipose carrier. In some cases, the adipose carrier includes a processed organic phase of decellularized adipose tissue that has been derived from a human donor and exposed to alkaline organic solution and that is substantially free of a stromal vascular fraction. In some cases, the bone component is at least partially demineralized. In some cases, the bone component includes cortical bone particles, cancellous bone particles, or a mixture thereof. In some cases, the adipose carrier is lyophilized.

Embodiments of the present invention provide adipose derived filler compositions that can be used to maintain a physical space upon implantation or administration to a patient treatment site.

In another aspect, embodiments of the present invention encompass compositions for treating a treatment site in a patient. Exemplary compositions may include a fibrous filler material derived from human donor decellularized adipose tissue. In some cases, a matrix resulting from processing the fibrous filler material provides a permanent structure. In some cases, the fibrous filler material is substantially free of oils or lipid content. In some cases, the fibrous filler material is freeze dried and shredded. In some cases, the fibrous filler material is combined with one or more space filling entities. In some cases, filler material can be present as a permanent structure having structural cavities that can be filled with a patient's own fat cells following implantation of the filler in the patient.

In another aspect, embodiments of the present invention include methods of manufacturing an adipose filler material for implantation into a patient. Exemplary methods may include obtaining adipose material that has been recovered from a human donor, dividing the adipose material into pieces, and separating the adipose material from oil, water, and debris. Optionally, methods may include wringing the adipose material pieces, for example to remove oil, water, and/or debris. In some cases, a process of harvesting adipose material from a donor may include recovering a full thickness of skin from the donor, where the full thickness of skin includes a fat portion and a skin portion. According to some embodiments, methods may include removing a skin portion from the harvested tissue. In some cases, methods may include isolating one or more sheets of fat fibers from the fat portion, where the adipose material includes one or more sheets of fat fibers. According to some embodiments, a step of isolating one or more sheets of fat fibers from the fat portion may include exposing the fat portion to a solution of sodium hydroxide and isopropanol. According to some embodiments, methods may include exposing the fat portion to the solution for between 15 and 45 minutes. In some cases, methods may also include exposing the fat portion to a phosphate buffered saline solution. In some cases, the separation step may include performing one or more freeze and thaw cycles. In some cases, one or more freeze and thaw cycles may include rapid freezing in liquid nitrogen and thawing in phosphate buffered saline. In some cases, methods may include freeze drying the pieces. In some cases, methods may include shredding the pieces. In some cases, methods may include exposing the pieces to one or more isopropanol wash and phosphate buffered saline wash cycles.

In yet another aspect, embodiments of the present invention may include methods of delivering an adipose filler material to a patient. Exemplary methods may include administering the adipose filler material to a treatment site of the patient, where the adipose filler material is derived from human donor decellularized adipose tissue. In some cases, a matrix resulting from processing the adipose filler material provides a permanent structure. In some cases, the adipose filler material is substantially free of oils or lipid content. In some cases, the patient treatment site is a void where surgical removal of tissue leaves a space that is not natural to the physiology of a removal site. In some cases, the adipose filler material can be administered as a space holder to separate a plurality of distinct surgical sites. In some cases, the adipose filler composition or material structure includes structural cavities that can be filled with a patient's own fat cells following implantation of the composition in the patient. In some cases, the adipose filler material is freeze dried. In some cases, the patient treatment site includes part of a reconstructive surgical location, and the adipose filler material provides a scaffold that significantly maintains a volume following implantation.

The above described and many other features and attendant advantages of embodiments of the present invention will become apparent and further understood by reference to the following detailed description when considered in conjunction with the accompanying drawings.

### BRIEF DESCRIPTION OF THE DRAWINGS

FIG. 1 illustrates aspects of adipose derived carrier compositions and methods of producing such compositions, including representative components of an implant carrier product, according to embodiments of the present invention.

FIG. 2 illustrates aspects of adipose derived carrier compositions and methods of producing such compositions, including aspects of a method for making a treatment product, according to embodiments of the present invention.

FIG. 3 illustrates aspects of adipose derived carrier compositions and methods of producing such compositions, including aspects of a three phase separation, according to embodiments of the present invention.

FIG. 4 illustrates aspects of adipose derived carrier compositions and methods of producing such compositions, including aspects of a three phase separation, according to embodiments of the present invention.

FIG. 5 illustrates aspects of adipose derived carrier compositions and methods of producing such compositions, including aspects of a three phase separation, according to embodiments of the present invention.

FIG. 6 illustrates aspects of adipose derived matrix compositions and methods of producing such compositions, according to embodiments of the present invention.

FIG. 7 illustrates aspects of adipose derived matrix compositions and methods of producing such compositions, according to embodiments of the present invention.

FIG. 8 illustrates aspects of adipose derived matrix compositions and methods of producing such compositions, including aspects of a three phase separation, according to embodiments of the present invention.

FIG. 9 illustrates aspects of adipose derived matrix compositions and methods of producing such compositions, including aspects of a three phase separation, according to embodiments of the present invention.

FIG. 10 illustrates aspects of adipose derived matrix compositions and methods of producing such compositions, including aspects of a three phase separation, according to embodiments of the present invention.

FIGS. 11 and 12 depict aspects of an exemplary allograft material and production process, according to embodiments of the present invention.

FIG. 13 depicts aspects of a method of producing an adipose derived fibrous filler material, according to embodiments of the present invention.

FIG. 14 depicts aspects of a method of producing an adipose derived fibrous filler material, including aspects of full thickness skin recovered from a donor, according to embodiments of the present invention.

FIG. 15 depicts aspects of a method of producing an adipose derived fibrous filler material, including the operation of a wringing device, according to embodiments of the present invention.

### DETAILED DESCRIPTION OF THE INVENTION

Embodiments of the present invention encompass adipose based compositions, and methods of their use and manufacture. Exemplary adipose compositions are well suited for use as carriers for various substances, as surgical reconstructive materials, as dermal fillers, and the like.

Adipose Derived Carrier Systems and Methods

Adipose tissue derived from a human donor can be processed to break down and/or disrupt an extracellular matrix (ECM) structure to provide a useful biological carrier for particles and other materials. Adipose derived tissue or material can operate as a putty, carrier, or glue, optionally for rendering granular particles into a moldable packable product. For example, a carrier derived from donor human tissue can be combined with cancellous and/or cortical bone particles, to form a putty or a paste. In some instances, a carrier can be combined with any tissue or material so as to improve or enhance the moldability or flowability of that tissue or material, for use as a scaffold for implantation at a treatment site within a patient, or as a fixative in non-weight bearing applications.

In some instances, adipose tissue is processed, optionally by decellularizing the tissue, to provide a putty, paste, or carrier. Processed adipose tissue can be combined with cancellous and/or cortical bone particles to provide a treatment composition for use with a patient. In some instances, bone particles included in the composition may have stem cells attached thereto. Stem cells can be obtained from adipose tissue recovered from an individual donor, as described elsewhere herein. Exemplary treatment compositions exhibit desirable moldability and packing characteristics, and may allow stem cell laden bone particles or other materials to be implanted at a treatment site within a patient and held in place during surgery. In some instances, adipose derived carrier material can be used to position loose particles as part of a surgical implantation procedure, and the patient's body can naturally remove the carrier subsequent to surgery.

According to some embodiments, the production of an adipose carrier involves processing fat or adipose tissue obtained from a human donor, and manipulating the concentrations of oils, moisture, and other components of the tissue so as to provide a carrier that can be easily mixed with particles and other substances. In use, the carrier and mixed particles remain in place at a treatment site, and are not easily washed away with irrigation. In some instances, compositions include adipose derived carrier and bone particles obtained from a common human donor. In some cases, the concentration of oils in the carrier or composition can be adjusted or selected so as to provide with stability at elevated temperatures that occur within a living human body.

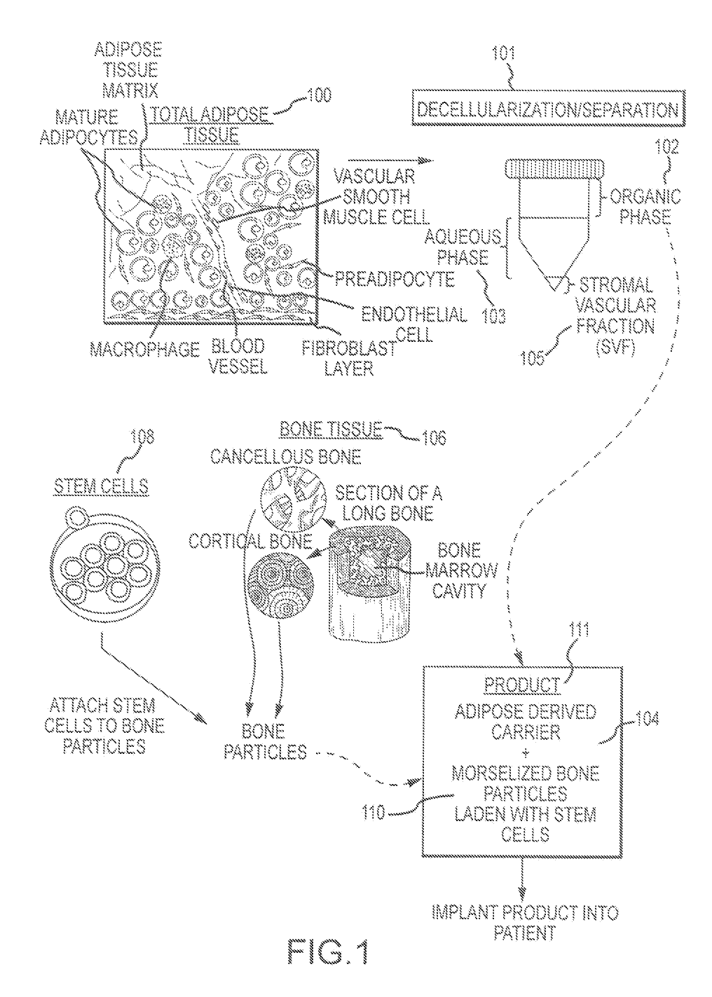

Turning now to the drawings, FIG. 1 depicts an aspect of a method for making an implant product according to embodiments of the present invention. Adipose tissue 100 may be obtained that has been recovered from a donor patient can be processed to obtain an organic phase material 102, which in turn can be processed to obtain an adipose derived carrier 104. It is understood that the adipose tissue matrix of adipose tissue 100 shown here is different from the processed adipose derived matrix material as discussed elsewhere herein. Often, the recovered adipose tissue 100 will have an extracellular matrix (ECM) with a particular three dimensional structure or architecture, which may include for example vascular structures, ductal structures, and the like. As discussed elsewhere herein, embodiments of the present invention encompass techniques for discomposing such three dimensional structures, whereby the organized architecture of the adipose extracellular matrix is transformed to a disorganized or random assortment of collagen strands and other extracellular matrix subunits. As shown here, according to a decellularization and separation process 101, the organic phase material 102 can be separated from an aqueous phase 103 and a stromal vascular fraction (SVF) 105. As shown here, the organic phase 102, aqueous phase 103, and SVF can be contained in a 250 ml centrifuge tube. Often, the SVF may include various components such as preadipocytes, mesenchymal stem cells, endothelial progenitor cells, T cells, B cells, mast cells, adipose tissue macrophages, and the like, and the organic phase material 102 may include adipose ECM components, such as collagen. Further, bone particles 106, and optionally stem cells 108, from a donor can be processed to obtain morselized bone particles laden with stem cells 110. According to some embodiments, the bone particles can include cancellous and/or cortical bone material. The adipose derived carrier 104 and stem cell laden bone particles 110 can be combined to produce a treatment composition product 111, which can then be implanted or administered at a treatment site of a patient. Optionally, the adipose derived carrier 104 can be combined with any desired material, which may include non-bone tissue, and/or non-tissue particles.

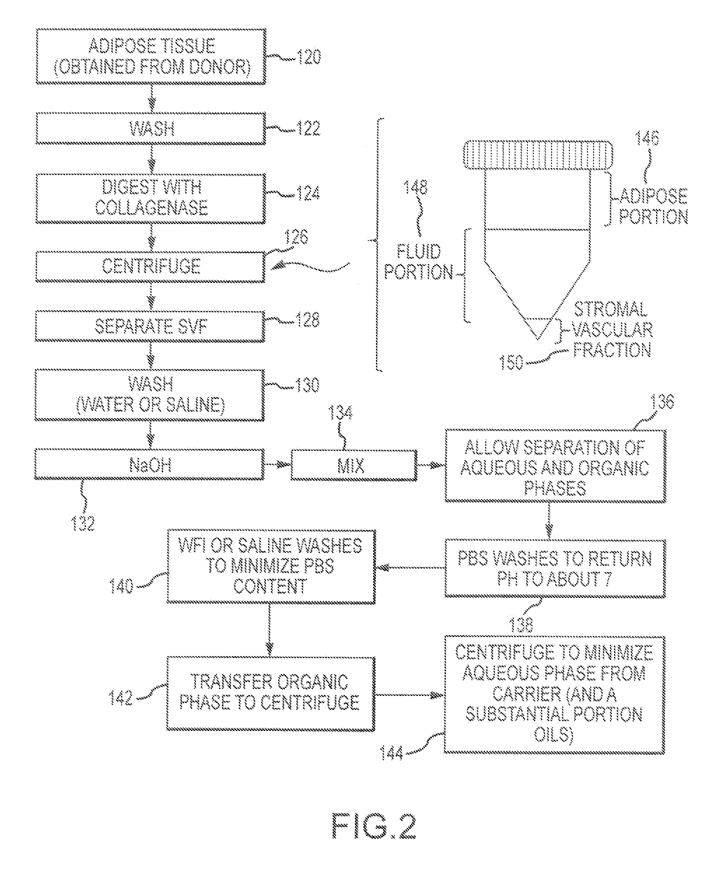

FIG. 2 depicts additional aspects of a method for making a treatment product according to embodiments of the present invention. As shown here, adipose tissue is recovered from a human donor, as indicated by step 120. In some cases, the adipose tissue is obtained from the human donor abdomen (e.g. abdominal fat). The adipose tissue is then washed (e.g. with phosphate buffered saline (PBS)), as depicted by step 122 and then digested with collagenase as indicated by step 124. Collagenase can operate to break down collagen within the adipose tissue, so as to facilitate release of stem cells and other materials from the adipose. The resulting material is then centrifuged as depicted by step 126 to provide an adipose portion 146, a fluid portion 148, and a stromal vascular fraction (SVF) 150. According to some embodiment, digestion of adipose to produce SVF can accomplished using methods such as those described in US Patent Publication No. 2010/0124776, incorporated herein by reference. The SVF 150 can be removed or separated from the adipose 146 and fluid portions 148 as indicated by step 128. The adipose portion 146 can include adipose ECM components, such as collagen. As described elsewhere herein, the SVF 150 can be used as a source for stem cells, which can be combined with bone particles and incorporated with an adipose derived carrier.

The adipose 146 and fluid 148 portions can be washed, for example with water or saline as depicted in step 130. As shown here, the adipose portion 146, fluid portion 148, and SVF can be contained in a 250 ml centrifuge tube. Extracellular matrix material from the adipose portion can treated with any of a variety of hydroxy forms of alkaline earth metals in solutions of alcohol of various polarities so as to disrupt the three dimensional structure of the adipose extracellular matrix. In some cases, the adipose ECM can be treated with an alkaline alcohol solution. In some cases, the adipose ECM can be treated with an alkaline organic solution. According to some embodiments, the adipose 146 and fluid 148 portions are processed with at least three equal volume washes with water for injection (WFI) or a 0.9 percent saline solution. The adipose, optionally along with the fluid portion can be treated with amounts of basic solution, for example an equal volume of a basic solution, such as sodium hydroxide, ethanol, methanol, isopropanol, benzene, potassium hydroxide, and calcium hydroxide. The basic solution can act to breakdown and/or disrupt the structure or architecture of the adipose ECM. For example, the basic solution can act to break down or decompose the macrostructure of ECM, producing a composition of randomly oriented pieces of ECM collagen strands. According to some embodiments, a solution of 1 M sodium hydroxide (NaOH) can be used to wash the adipose and fluid portions as indicated by step 132 with mixing as indicated by step 134. In some cases, step 132 may involve a wash using any of a variety of hydroxy forms of alkaline earth metals in solutions of alcohol of various polarities, an alkaline alcohol solution, or an alkaline organic solution, so as to disrupt the three dimensional structure of the adipose extracellular matrix. The mixture that results from step 134 includes randomly oriented strands of collagen from the ECM, along with oils (e.g. lipids), and moisture. According to some embodiments, the mixture may be present as an emulsion. Organic and aqueous phases are then allowed to separate as indicated by step 136.

With regard to collagen fibers which are present in the adipose ECM, such fibers are typically composed of multiple collage fibrils, and such fibrils in turn are typically composed of triple-helix collagen molecules. Further, such triple-helix collagen molecules are in turn composed of single helix collagen polypeptide chains or strands. In some cases, collagenase treatments as discussed herein may operate to disrupt or cleave the single helix collagen polypeptide at a location along the chain or strand. In some cases, alkaline alcohol solution, alkaline organic solution, or basic solution treatments as discussed herein may operate to disrupt the triple-helix collagen structure, for example by breaking bonds which are present between adjacent single chains or strands of the triple-helix. According to some embodiments, collagenase may operate as a non-specific enzyme for collagen, thus degrading the collagen generally. In some cases, collagenase may operate to attack certain sites in the collagen molecule. With the —OH (hydroxide radical in alkaline solution), the attack can be even less specific. According to some embodiments, the collagenase can operate in a more mild fashion in disrupting collagen structure, whereas the hydroxide in alkaline solution can be less specific and operate in a rougher fashion, and more quickly than the collagenase. Hence, for example, both the collagenase and the alkaline solution (e.g. alcohol or organic) can operate to degrade collagen In some cases, the collagenase degrades collagen more slowly, whereas the hydroxide degrades collagen more quickly. In some instances, a tropocollagen or collagen molecule is a subunit of larger collagen aggregates such as fibrils and as such any process that disrupts collagen structures will disrupt the tropocollagen bands. According to some embodiments, the byproduct of this disruption is polypeptide chains. Such chains may or may not retain helical structure, and the structure may depends on the chain length and the amino acid sequence. According to some embodiments, when preparing the adipose carrier or matrix materials, there may be no emphasis on maintaining helical structures of the collagen chains or strands. Relatedly, there may be no emphasis on identifying the length or composition of the polypeptide chain fragments. According to some embodiments, adipose carrier or matrix materials include a mixture of single chain collagen polypeptides or fragments thereof, triple-helix collagen molecules or fragments thereof, and collagen fibrils or fragments thereof, and other combinations involving such components.

As depicted in step 136, methods may involve allowing organic and aqueous phases to separate. In some cases, the organic phase can include short chain lipids, long chain lipids, debris, and extracellular components such as collagen. In some cases, the lipids which remain associated with the collagen are less polar than the alcohol or organic solution used to treat the collagen (e.g. in step 132). For example, if the lipids are less polar than the alcohol or organic solution used, then the lipids may stay in the organic phase with the collagen. In some cases, the aqueous phase can include NaOH (or other alcohols), blood, and debris.

Following separation, the phases can be processed with at least two washes of equal volume of PBS to return or to adjust the pH of the solution to about 7.0 as indicated at step 138. Subsequently, the material can be processed with at least three more washes with WFI or a 0.9% saline as shown in step 140, so as to minimize or reduce the PBS content. As shown in step 142, the organic phase can be transferred to a centrifuge, and spun down to minimize or further reduce any amount of aqueous phase from the carrier material, as well as a substantial portion of the oils as shown in step 144. Carrier materials produced according to such protocols typically include a random mixture of disrupted collagen strands, optionally along with an amount of lipids and moisture. The carrier can be mixed with morselized cancellous and/or cortical bone to provide a moldable and packable composition. In some cases, the carrier can be mixed with partially demineralized cancellous bone particles laden with adipose derived mesenchymal (adult) stem cells. The bone and/or stem cells may be obtained from the same donor from which the adipose derived carrier is obtained. In use, the mesenchymal stem cells placed or seeded onto the bone particles may be signaled to become osteoblasts. In turn, the osteoblasts can respond to biological signaling to form natural bone. Optionally, the carrier can be mixed with any desired granular product.

In one example, adipose tissue was processed to remove the SVF (e.g. with collagenase digestion). Following removal of the SVF, the remaining material (e.g. remaining adipose and aqueous portions similar to what is depicted in FIG. 2) was subjected to an extraction or fractionation process. Aqueous materials were removed by adding either WFI or 0.9% saline into a 500 ml reparatory funnel. The contents were shaken, and separation between the organic and aqueous phases was allowed to occur. It was observed that saline solution appeared to remove a greater amount of debris and color (reddish) from the fat, as compared with the water for injection. Solids were precipitated via various protocols, for example by adding a 70% isopropanol (IPA), 1 M hydrochloric acid (HCl), and/or concentrated NaOH.

The 70% IPA treatment produced a three phase separation as follows. The bottom layer included an aqueous phase, the middle layer included an organic phase, and the top layer included a solids phase. It was observed that the IPA was slightly cloudy, in the organic phase.

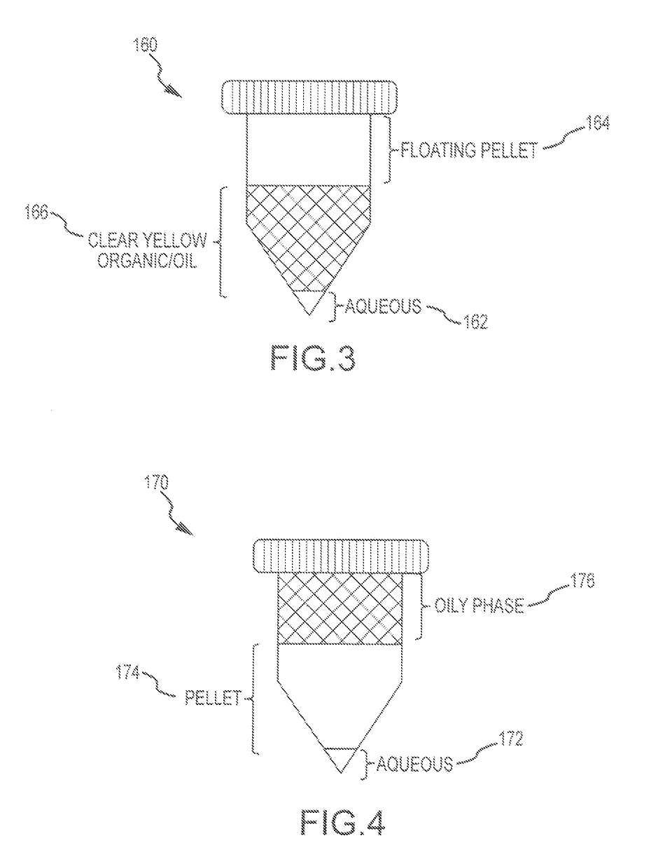

The 1 M HCl treatment produced a two phase separation as follows. The bottom layer included an aqueous phase with the bulk of HCl, and the top layer included an organic phase of a solid that was suspended therein. Optionally, this extraction involved removing the aqueous phase and neutralizing the pH in the organic phase by washing with two equal volumes of PBS followed by two washes of 0.9% saline to remove as much PBS as possible. Once the organic phase was washed, the organic phase was transferred into 50 ml conical tubes and centrifuged at 475 g for 15 minutes. The spun down solution 160 included three phases, as schematically depicted in FIG. 3. As shown here, the pellet 164, organic/oil portion 166, and aqueous portion 162 can be contained in a 50 ml centrifuge tube. A 45 ml sample yielded about 5 ml of aqueous phase 162, about 5 ml of floating pellet 164, and about 35 ml of clear yellow oil 166. Floating pellets 164 were collected and combined. The pellets 164 were oily and when mixed behaved like a paste (e.g. similar to peanut butter). The pellets 164 contained disrupted ECM collagen strands.

The concentrated NaOH treatment produced a final-spin three phase separation 170 as follows. The bottom layer included an aqueous phase 172, the middle layer included a pellet material 174, and the upper layer included an oily phase 176, as schematically illustrated in FIG. 4. As shown here, the pellet 174, organic/oil portion 176, and aqueous portion 172 can be contained in a 50 ml centrifuge tube. A 45 ml sample yielded about 5 ml of aqueous phase 172, about 5 ml of pellet 174, and about 35 ml of oily phase 176. The pellet material 174 was observed to similar in type to that of the HCl treatment.

In another process, solids were precipitated from processed adipose tissue by adding an equal volume of 70% IPA with 0.1 N NaOH with mixing, and then allowing separation of the aqueous and organic phases. It is understood that where normality (N) is used to characterize NaOH, that other measures of concentration may be used. For example. 0.1 N NaOH has the same concentration as 0.1 M NaOH.

Alternative NaOH treatment protocols using different concentrations were performed. The organic phase from a new batch of processed fat (with SVF removed) was subjected to a less concentrated 1 N NaOH treatment. In the final spin, the amount of precipitated pellet was observed to be significantly larger than with the more concentrated NaOH treatment. For example, the amount of pellet was more than 50% of the volume centrifuged. An upper layer included about 5 to 7 ml of oil, the middle layer included about 20 to 25 ml of pellet, and the lower layer included about 10 ml of aqueous phase. The pellet material was removed, and it was observed to have a runny consistency. Approximately 1.5 ml of pellet material was placed in a 2 cc eppendorf tube and ultracentrifuged at 13,000 rpm for 3 minutes. A three phase separation was observed as follows. The lower layer included aqueous phase, the middle layer included pellet material, and the upper layer included an oil phase. The observed ratio of pellet was 50% or more, and oil and aqueous phases were still present. Extracted pellet material from multiple tubes was combined, and the resulting paste/putty composition was observed to be creamy. The composition was observed to mix well with moist cancellous/morselized bone chips, and the combination held together very well. The extracted fat carrier material appears to be of an ideal consistency to produce a stem cell putty.

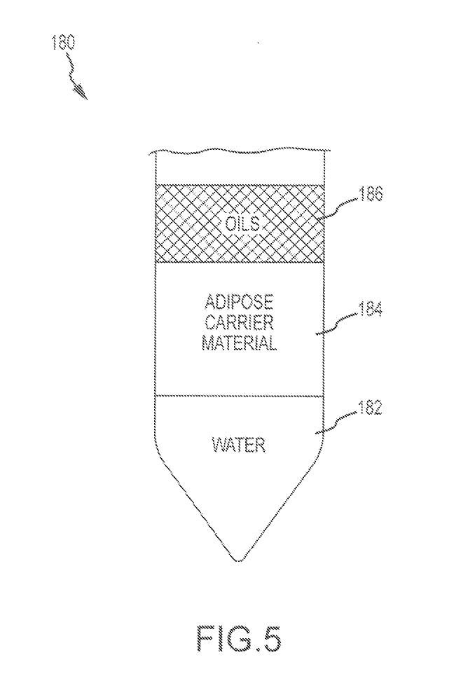

In another example, an organic paste was created that can be used with a morselized bone material (optionally containing stem cells) to provide a malleable composition which can be implanted or administered to a patient by a physician or doctor. Digested fat material (obtained from an SVF removal procedure, as described elsewhere herein) was washed with cold sodium chloride (NaCl) until the precipitate was clear (about 3 to 4 washes). Subsequently, 1 N NaOH (equal volume to last amount of NaCl removed) was added. Without being bound by any particular theory, it is thought that the NaOH (or other basic solutions, alkaline alcohol solutions, or alkaline organic solutions) may help denature the adipose proteins even further. The NaOH was allowed to sit with the material for about 10 to 15 minutes, and then the liquid was precipitated out. Two to three more NaCl washes were performed. The remaining adipose mixture was loaded into a 50 ml conical centrifuge tube, and centrifuged for 15 minutes at maximum speed. A three phase separation 180 was observed as follows. The lower layer included a water phase 182, the middle layer included an adipose carrier phase 184, and the upper layer included an oils phase 186, as schematically illustrated in FIG. 5. As shown here, the carrier phase 184, organic/oil portion 186, and aqueous portion 182 can be contained in a 2 ml centrifuge tube. The oil 186 and water layers 182 were decanted from the conical tube. The remaining adipose carrier material 184 was loaded into 2 ml eppendorf tubes and centrifuged at a higher rpm to separate out even more oil and water. Subsequently, oil and water were decanted from the 2 ml tubes. As a result, the remaining adipose carrier material 184 had a consistency of whipped butter. When added to a morselized bone product (e.g. AlloStem®, or adipose derived mesenchymal adult stem cells combined with partially demineralized cancellous bone) it was observed that the adipose carrier material helped the morsels to stick together, so as to create a very malleable paste/putty like substance.

Adipose Derived Matrix Systems and Methods

Adipose tissue derived from a human donor can be processed to breakdown and/or disrupt an ECM structure to provide a useful biological matrix for cells, proteins, large molecules, and other materials. In some instances, the cells, proteins, large molecules, or other materials may be present in a solution, which is then combined with or absorbed by the matrix. In this way, the matrix can operate as a vehicle for incorporated materials. Often, a matrix material may have a talc like powder consistency. For example, an adipose derived matrix, upon absorbing a solution which contains cells, proteins, and/or other large molecules, may result in a putty or gel. Subsequently, the putty or gel can be delivered to treatment site of a patient as a putty or injectable gel. In some instances, an adipose derived matrix can be combined with mesenchymal stem cells, so as to provide an injectable form of stem cell therapy. In some instances, an adipose derived matrix can be provided in a dry powder form, which can be combined with a therapeutic in liquid form, and the combined matrix and therapeutic can be injected into a treatment site of a patient. In some instances, an adipose derived matrix may be provided as a dry, decellularized, and/or aseptic, adipose matrix.

In some instances, an adipose matrix material is provided as an allogeneic delivery vehicle. Embodiments of the present invention encompass pure, clean matrix materials, which are derived from a tissue that is common to the vast majority of the human body. Hence, the matrix materials are usefully applicable to a wide range of injured/surgical sites. Method of preparing a matrix material can reduce the possibility of rejection by the recipient.

In some instances, adipose tissue is processed, optionally by decellularizing the tissue, to provide a putty, gel, or powder. Processed adipose tissue can be combined with cells, proteins, and/or large molecules to provide a treatment composition for use with a patient. In some instances, adipose derived matrix material can be used to maintain selected therapeutics at a treatment site within a patient, for an extended duration of time.

According to some embodiments, the production of an adipose matrix involves processing fat or adipose tissue obtained from a human donor, and manipulating the concentrations of oils, moisture, and other components of the tissue so as to provide a matrix that can be combined with other materials.

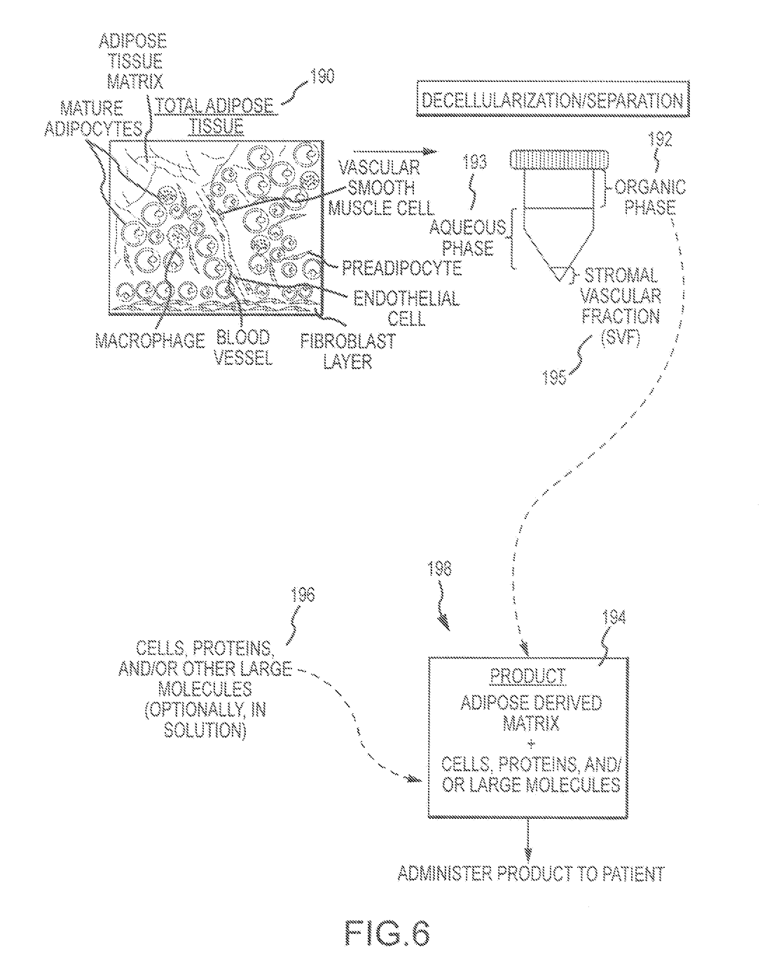

Turning now to the drawings, FIG. 6 depicts an aspect of a method for making a matrix material according to embodiments of the present invention. Adipose tissue 190 may be obtained that has been recovered from a donor patient can be processed to obtain an organic phase material 192 (e.g. by separation from an aqueous phase 193 and a stromal vascular fraction 195), which in turn can be processed to obtain an adipose derived matrix 194. It is understood that the adipose tissue matrix of adipose tissue 190 shown here is different from the processed adipose derived matrix material as discussed elsewhere herein. As shown here, the organic phase 192, aqueous phase 193, and SVF 195 can be contained in a 250 ml centrifuge tube. As with the carrier embodiments discussed above, the recovered adipose tissue can include an extracellular matrix material having a three dimensional structure or architecture. In some instances, the matrix material 194 includes disrupted collagen strands derived from adipose ECM. Further, cells, proteins, and/or large molecules 196, optionally in solution, can be combined with the matrix to produce a treatment composition product 198, which can then be implanted or administered at a treatment site of a patient. Optionally, the adipose derived matrix 194 can be combined with any desired material, such as mesenchymal stem cells, Platelet-Rich Plasma, and the like. In some embodiments, matrix material can be combined with bone material (e.g. AlloStem®, or adipose derived mesenchymal adult stem cells combined with partially demineralized cancellous bone).

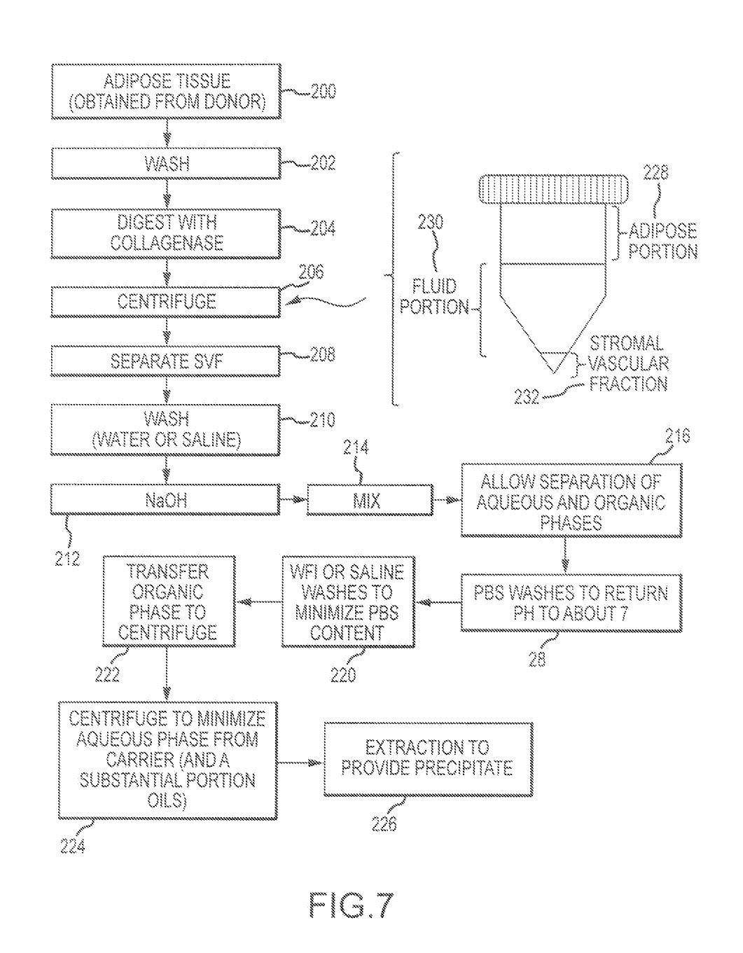

FIG. 7 depicts additional aspects of a method for making a treatment product according to embodiments of the present invention. As shown here, adipose tissue is recovered from a human donor as depicted in step 200. In some cases, the adipose tissue is obtained from the human donor abdomen (e.g. abdominal fat). The adipose tissue is then washed (e.g. with PBS) as depicted in step 202 and then digested with collagenase as depicted in step 204. Collagenase can operate to break down collagen within the adipose tissue, so as to facilitate release of stem cells and other materials from the adipose.

In some cases, following the collagenase digestion, the adipose tissue can be further washed, for example with at least three equal volume washes with either water for injection or 0.9% saline solution. Further optionally, the adipose tissue can be processed by adding an equal volume of 70% IPA with 0.1 N NaOH with mixing, and then allowing separation of the aqueous and organic phases.

In some cases, following the collagenase digestion, the resulting material is then centrifuged to provide an adipose portion 228, a fluid portion 230, and a SVF 232 as depicted in step 206. As shown here, the adipose portion 228, fluid portion 230, and SVF 232 can be contained in a 250 ml centrifuge tube. The SVF 232 can be removed or separated from the adipose and fluid portions as depicted in step 208. As described elsewhere herein, the SVF 232 can be used as a source for stem cells, which can be incorporated into the adipose derived matrix, optionally along with cells, proteins, and/or other large molecules. The adipose 228 and fluid portions 230 can be washed, for example with water or saline as depicted in step 210. According to some embodiments, the adipose and fluid portions are processed with at least three equal volume washes with WFI or a 0.9 percent saline solution, followed by an equal volume of a basic solution, such as sodium hydroxide, ethanol, methanol, isopropanol, benzene, potassium hydroxide, and calcium hydroxide. The basic solution can act to breakdown and/or disrupt an ECM structure within the adipose tissue. For example, a solution of 1 N NaOH as depicted in step 212 with mixing as depicted in step 214. Organic and aqueous phases are then allowed to separate as depicted in step 216.

Following separation, the phases can be processed with at least two washes of equal volume of PBS to return or adjust the pH of the solution to about 7.0 at block 218. Subsequently, the material can be processed with at least three more washes with WFI or a 0.9% saline, so as to minimize or reduce the PBS content as depicted in step 220. The organic phase can be transferred to a centrifuge as depicted in 222, and spun down to minimize or further reduce any amount of aqueous phase from the matrix material, as well as a substantial portion of the oils as depicted in step 224. According to some embodiments, any of the above described steps for FIG. 7 can be replaced with related steps shown in FIG. 2.

Subsequently, extraction with a polar solution, such as 1-propanol, can be performed via centrifugation to remove all or substantially all oils and aqueous content, leaving a white precipitate with is then filtered and washed with water as depicted in step 226. Other polar solutions, such as ethanol and acetone may also be used in the extraction of the matrix. In some cases, the extraction is performed exhaustively. The retentate can then be freeze dried to a powder to provide the matrix. In some cases the matrix is provided as a powder with a very fine grain size.

In one example, adipose tissue was processed to remove the stromal vascular fraction (e.g. with collagenase digestion). Following removal of the SVF, the remaining material was subjected to an extraction or fractionation process. Aqueous materials were removed by adding either WFI or 0.9% saline into a 500 ml reparatory funnel. The contents were shaken, and separation between the organic and aqueous phases was allowed to occur. It was observed that saline solution appeared to remove a greater amount of debris and color (reddish) from the fat, as compared with the water for injection. Solids were precipitated by adding a 70% IPA, 1 M HCl, and concentrated NaOH.

The 70% IPA treatment produced a three phase separation as follows. The bottom layer included an aqueous phase, the middle layer included an organic phase, and the top layer included a solids phase. It was observed that the IPA was slightly cloudy, in the organic phase.

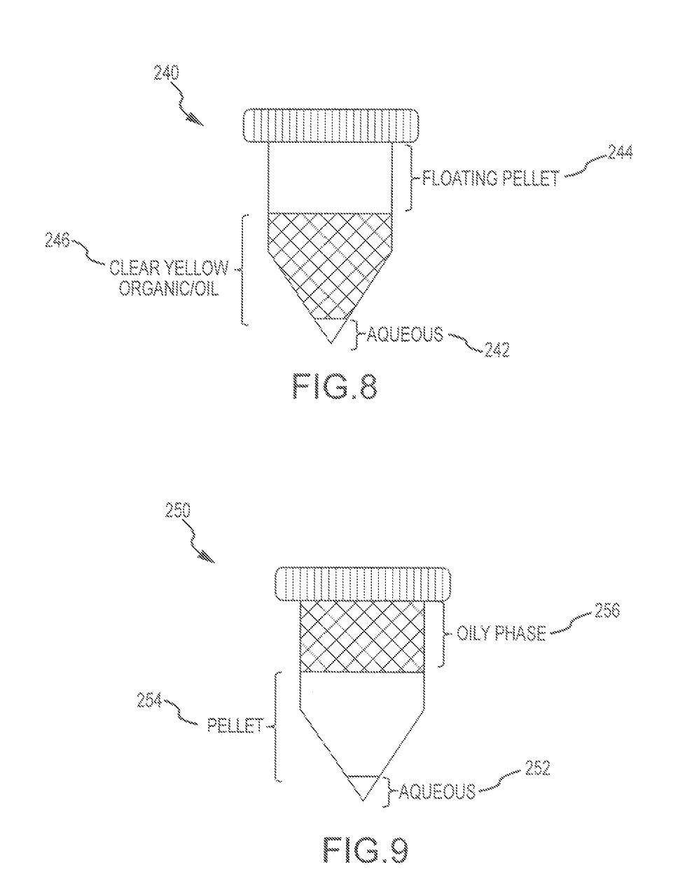

The 1 M HCl treatment produced a two phase separation as follows. The bottom layer included an aqueous phase with the bulk of HCl, and the top layer included an organic phase of a solid that was suspended therein. Optionally, this extraction involved removing the aqueous phase and neutralizing the pH in the organic phase by washing with two equal volumes of PBS followed by two washes of 0.9% saline to remove as much PBS as possible. Once the organic phase was washed, the organic phase was transferred into 50 ml conical tubes and centrifuged at 475 g for 15 minutes. The spun down solution 240 included three phases, as schematically depicted in FIG. 8. As shown here, the pellet 244, organic/oil portion 246, and aqueous portion 242 can be contained in a 50 ml centrifuge tube. A 45 ml sample yielded about 5 ml of aqueous phase 242, about 5 ml of floating pellet 244, and about 35 ml of clear yellow oil 246. Floating pellets 244 were collected and combined. The pellets 244 were oily and when mixed behaved like a paste (e.g. similar to peanut butter). Next, exhaustive extraction with 1-propanol or other polar solution can be performed via centrifugation to remove all or substantially all oils and aqueous content, leaving a white precipitate which can be filtered and washed with water. The retentate can be freeze dried to a dry powder.

In related embodiments, a concentrated NaOH treatment can produce a final-spin three phase separation 250 as follows. The bottom layer includes an aqueous phase 252, the middle layer includes a pellet material 254, and the upper layer includes an oily phase 256, as schematically illustrated in FIG. 9. As shown here, the pellet 254, organic/oil portion 256, and aqueous portion 252 can be contained in a 50 ml centrifuge tube. A 45 ml sample can yield about 5 ml of aqueous phase 252, about 5 ml of pellet 254, and about 35 ml of oily phase 256. The pellet material 254 can be similar in type to that of the HCl treatment. Next, exhaustive extraction with 1-propanol or other polar solution can be performed via centrifugation to remove all or substantially all oils and aqueous content, leaving a white precipitate which can be filtered and washed with water. The retentate can be freeze dried to a dry powder.

Alternative NaOH treatment protocols using different concentrations can be performed. The organic phase from a new batch of processed fat (with SVF removed) can be subjected to a less concentrated 1 N NaOH treatment. In the final spin, the amount of precipitated pellet may be observed to be significantly larger than with the more concentrated NaOH treatment. For example, the amount of pellet can be more than 50% of the volume centrifuged. An upper layer may include about 5 to 7 ml of oil, the middle layer may include about 20 to 25 ml of pellet, and the lower layer may include about 10 ml of aqueous phase. The pellet material can be removed, and it may be observed to have a runny consistency. Approximately 1.5 ml of pellet material can be placed in a 2 cc eppendorf tube and centrifuged at 13,000 rpm for 3 minutes. A three phase separation may be observed as follows. The lower layer may include an aqueous phase, the middle layer may include pellet material, and the upper layer may include an oil phase. An observed ratio of pellet 50% or more, and oil and aqueous phases may still be present.

The description above can relate to a carrier composition that is produced prior to the derivation of an adipose matrix. This carrier composition can have the same characteristics and can be attained using the same techniques as described in the carrier section of the application. Additionally, any of the techniques or described in the carrier portion of the matrix section may be applied to the carrier section of the application. Hence, features of the described carrier embodiments are applicable to the described matrix embodiments, mutatis mutandis, and vice versa.

Next, exhaustive extraction with 1-propanol or other polar solution can be performed via centrifugation to remove all or substantially all oils and aqueous content, leaving a white precipitate which can be filtered and washed with water. The retentate can be freeze dried to a dry powder.

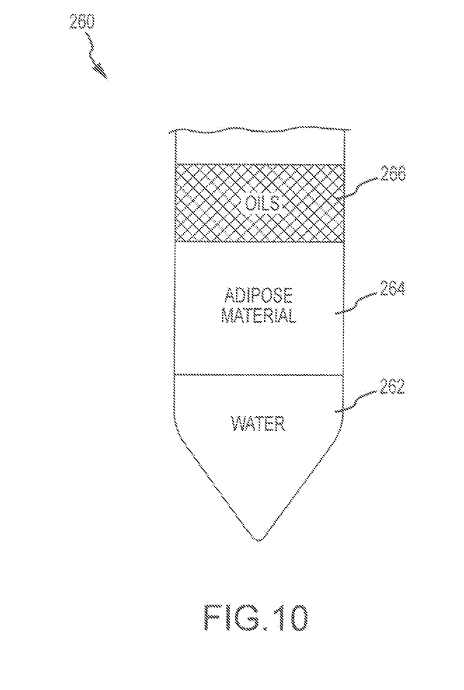

In another example, digested fat material (obtained from an SVF removal procedure, as described elsewhere herein) can be washed with cold NaCl until the precipitate is clear (e.g. about 3 to 4 washes). Subsequently, 1 N NaOH (equal volume to last amount of NaCl removed) can be added. Without being bound by any particular theory, it is thought that the NaOH may help denature the adipose proteins even further. The NaOH can be allowed to sit with the material for about 10 to 15 minutes, and then the liquid can be precipitated out. Two to three more NaCl washes can be performed. The remaining adipose mixture can be loaded into a 50 ml conical centrifuge tube, and centrifuged for 15 minutes at maximum speed. A three phase separation 260 may be observed as follows. The lower layer may include a water phase 262, the middle layer may include an adipose material phase 264, and the upper layer may include an oils phase 266, as schematically illustrated in FIG. 10. As shown here, the adipose material 264, organic/oil portion 256, and aqueous portion 262 can be contained in a 2 ml centrifuge tube. Next, exhaustive extraction with 1-propanol or other polar solution can be performed via centrifugation to remove all or substantially all oils and aqueous content, leaving a white precipitate which can be filtered and washed with water. The retentate can be freeze dried to a dry powder. Such powder can include small particles of disrupted adipose ECM collagen.

According to some embodiments, any of the dry powders described above can be combined with a concentrated mesenchymal stem cell slurry and then cryopreserved, optionally for long term storage. At the point of use (e.g. surgical operating room), the mixture can be rinsed free of the cryopreservation medium and then either applied via a spatula or loaded onto a syringe and injected to the site. The matrix can provide the mesenchymal stem cells with a natural environment for storage. Further, the matrix can be easily broken down at the therapeutic site to be remodeled to the correct tissue where it resides.

According to some embodiments, the dry matrix is supplied as a powder is combined with a therapeutic slurry of the type, such as Platelet-Rich Plasma (PRP), which allows for the slurry to remain at the implant site for a prolonged amount of time compared to injecting PRP by itself to the site.

Relatedly, the dry matrix can be used with any injectable product or therapeutic that can benefit from a long or extended residence time at an injection or treatment site.

Mesenchymal Stem Cell Composition Systems and Methods

Embodiments of the present invention encompass compositions, including bone graft materials, which may include an adipose derived carrier combined with cells. In some cases, embodiments encompass tissue composites containing bone, stem cell, and adipose components. In some cases, stem cell components may be provided as mesenchymal stem cells (MSC's), which have the ability to differentiate into a variety of different cells, including adipose, bone, and cartilage. The tissue composite material can be cryopreserved for packaging, storage, transport, and/or later use. Assays such as cell counting kit-8 (CCK-8) can be used to evaluate or verify post-cryopreservation viable cell counts. Exemplary compositions are well suited for use in bone treatments. For example, the bone component (e.g. cancellous bone, optionally at least partially demineralized) can help to facilitate osteoconduction by providing a scaffold to promote new bone growth. Further, the tissue composition can help to facilitate osteoinduction by providing signaling molecules to stimulate new bone formation. What is more, the tissue composition can help to facilitate osteogenesis, whereby the stem cells differentiate into osteoblasts which form new bone. Optionally, the stem cells can differentiate into other cell types, such as cartilage (e.g. via chondrogenesis), adipose (e.g. via adipogenesis), skin, and the like. In some cases, the tissue composition can be provided as a sticky or adherent material, such as a gel or a putty, which does not move or wash away when placed at a treatment site of a patient. The material can be molded or formed, and applied to various patient anatomical structures or spaces, or in combination with implant devices such as cages and the like. In some cases, the tissue composition can operate to help absorb fluids such as blood and serum at the treatment site.

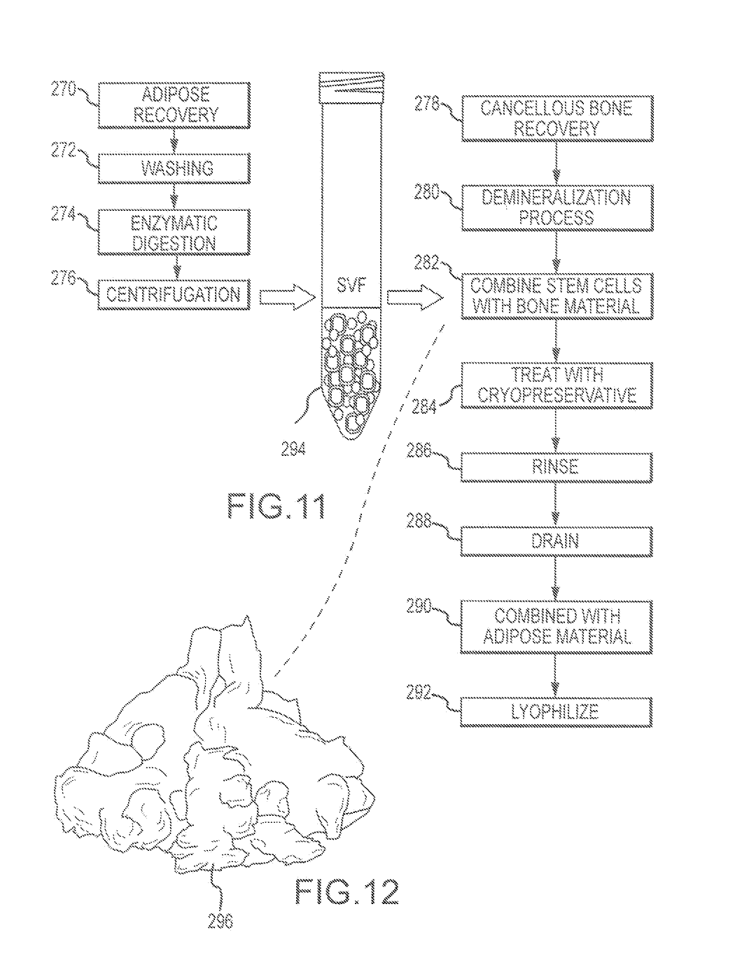

Turning now to the drawings, FIG. 11 illustrates aspects of a tissue composition manufacturing process, according to embodiments of the present invention. As shown here at step 270, adipose tissue can be obtained that has been recovered from a donor, optionally from abdominal fat obtained from the individual. For example, the manufacturing process may involve recovering 2000 ml of adipose. The adipose is then rinsed, for example with a PBS as depicted in step 272. Further, an enzymatic digestion material (such as collagenase) can be added, so as to help break down collagen present in the adipose, and thus release stem cells from the adipose as depicted in step 274. Upon digestion, the material may be centrifuged as depicted in step 276, which can operate to isolate stem cells toward the bottom of a centrifuge tube (e.g. 10 ml tube) as a pellet. As shown here, the SVF 294 may include stem cells. In some cases, stem cells may be obtained that have been recovered from other tissues, such as umbilical cord material or dental tissue material, obtained from a donor.

Exemplary techniques can also include obtaining bone tissue that has been recovered from a donor as depicted in step 278, and subjecting the bone to a demineralization process as depicted in step 280. Often, the bone is processed into small particles such as morsels. Stem cells recovered from adipose or other tissue can be combined with the bone material as depicted in step 282. For example, the stem cells can be seeded onto the bone material, optionally incubated for a period of 36 hours. Thereafter, the combined stem cell and bone material may be rinsed. Exemplary stem cell and bone material compositions and techniques are described in Shi et al., “Adipose-derived stem cells combined with a demineralized cancellous bone substrate for bone regeneration” Tissue Eng. Part A, July, 18 (13-14): 1313-21 (2012), the content of which is incorporated herein by reference. FIG. 12 depicts an exemplary material 296 containing combined stem cell and bone material, in a morselized form. In some cases, the combined stem cell and bone material is rinsed, for example so as to wash away other cells, such as blood cells.

As illustrated in FIG. 11, the combined stem cell and bone material can be treated with a cryopreservative as depicted in step 284 (e.g. without fully cryopreserving the combined step cell and bone material), rinsed as depicted in step 286, combined with an adipose carrier or matrix material obtained from adipose as depicted in step 290, cryopreserved as depicted in step 292, and packaged. In some cases, treated stem cell and bone material can be added to or mixed with an adipose material which has been lyophilized.

In some cases, the cryopreservative treatment protocol may include exposing the combined stem cell and bone material to a cryopreservation agent or cryoprotectant, such as dimethyl sulfoxide (DMSO) as depicted in step 284. Various solutions may be used, for example 5% DMSO, 10% DMSO, 20% DMSO, and the like. Often, the cryopreservation protocol will involve exposing the stem cell and bone material to the cryoprotectant for only a brief amount of time. For example, the stem cell material may be exposed to the cryoprotectant for about 5 seconds or less. In some cases, the exposure is about 4 seconds. In some cases, the exposure is about 3 seconds. In some cases, the exposure is about 2 seconds. In some cases, the exposure is about 1 second. The cryoprotectant exposure step can be carried out at room temperature.

Following the cryoprotectant solution exposure step as depicted in step 284, the cryoprotectant can be removed or washed away (e.g. with a rinse solution such as saline) as depicted in step 286. Hence, some amount of cryoprotectant may remain associated with the stem cell and bone material (e.g. DMSO absorbed into stem cells, where it operates to prevent crystal formation therein at low temperatures). The excess cryoprotectant (e.g. DMSO which is not in the stem cells or bone material), however, can be washed away. Following the rinse as depicted in step 286, the material may then be drained as depicted in step 288, for example by placing the material in a sieve. At this point, there may be only small amounts (e.g. less than 50 ppm) of cryoprotectant (e.g. DMSO) in the rinsed and drained stem cell and bone material. Typically, the amount of cryoprotectant, such as DMSO, will be present at levels which are acceptable for injection or administration to a patient. Upon draining the stem cell and bone material, which may be present as morsels, CAN remain hydrated, yet excess fluid is drained away. According to some embodiments, step 284 involves a limited exposure of seeded MSC's to DMSO to absorb sufficient DMSO into the cell structure to preserve the cells. As explained elsewhere herein, the MSC's can then be mixed with adipose carrier, optionally followed by a final freezing. Hence, step 284 may involve more of an exposure process, as compared with a true cryopreservation process.

As shown in FIG. 11, the drained stem cell and bone material can be mixed with an adipose material as depicted in step 290. Exemplary adipose materials (e.g. carrier or matrix) for use in combination with the stem cell and bone material are described in U.S. Ser. No. 61/684,386 filed Aug. 17, 2012 and U.S. Ser. No. 61/715,969 filed Oct. 19, 2012, which are incorporated herein by reference. In some cases, one or more of the combined components (stem cell, bone, and/or adipose) can be obtained from the same donor or individual.

The combined material can then be lyophilized as depicted in step 292. In some cases, the material can be cryopreserved in liquid nitrogen. The combined material can be stored at −80° C., for example. In some cases, the adipose carrier or material is lyophilized, and the lyophilized adipose carrier or material is then combined with the treated (e.g. exposed to DMSO) stem cell and bone material which has not been lyophilized. Hence, the stem cells may not be lyophilized in some embodiments. In many cases, the composite graft material is placed in a suitable container or package.

Accordingly, embodiments of the present invention encompass production methods which include combining stem cell and bone morsels, and exposing the material to a cryopreservation solution. The solution is allowed to be in contact with the morsels for a brief amount of time, and is then drained from the morsels and rinsed. For example, two volumes of saline solution can be used to wash off any excess cryopreservation solution. The morsels are then allowed to drain, and can then be combined with adipose carrier or matrix.

In some cases, the adipose material can be derived from an earlier step in the process where the MSC's are extracted from the adipose. The MSC free adipose material can be washed, decellularized with basic IPA, and pH neutralized with PBS. The adipose material can be separated from the aqueous content and from lipid oils by centrifugation.

The combined stem cell, bone, and adipose material can be aliquoted, placed in an appropriate container, double bagged, and frozen to −80° C. for long term storage. When the product is selected for use, it can then be thawed and implanted.

It has been observed that upon testing, exemplary adipose material shows no toxicity in vitro and in vivo, upon testing for cytotoxicity to cellular and tissue exposure. Exemplary adipose material has also been evaluated for any interference in the growth of new bone in an athymic mouse, and was observed to not interfere or promote growth.

In use, the combined stem cell, bone, and adipose material can be used for tissue repair and other medically beneficial applications. Mesenchymal stem cells or multipotent stromal cells within the combined material can differentiate into osteoblasts (bone), chondrocytes (cartilage), adipocytes (fat), and the like. In some cases, the combined material may be used as a stem cell bone growth substitute. In some cases, the bone component may be present as partially demineralized cancellous bone.

According to some embodiments, the composite stem cell, bone, and adipose product can be provided as a putty-like material composition, which contains MSC's, in a ready-to-use (after thawing) format. In some cases, composite stem cell, bone, and adipose product is provided as an allograft in putty or gel form, and does not require washing away of excess cryopreservation solution at the point of use (e.g. due to the brief exposure and/or draining and rinsing steps discussed elsewhere herein). The composite material is well suited for use for orthopedic indications. Hence, embodiments of the present invention encompass procedures by which a tissue containing MSC's can be cryopreserved without excess cryopreservation solution. Along with a reduction in the amount of cryopreservation solution present, an MSC loaded allograft can be combined with an adipose material (e.g. an adipose derived carrier as disclosed elsewhere herein), resulting in a composite product with beneficial moldability and packability characteristics. Accordingly, the composite product can be provided as a cryopreserved tissue with viable MSC's, which is ready to implant upon thawing as a putty or gel. In this way, MSC loaded morsels (or other dimensional grafts such as dowels, rods, blocks, strips, and the like) can be preserved with no excess cryopreservation solution in the composite product. What is more, the composite product may not require an involved and lengthy preparation process at the point of use, prior to implantation. Rather, the product can be thawed and implanted or administered to a patient, upon thawing. Relatedly, the composite product can be provided as a free flowing moldable putty, and can be molded and packed into the treatment site without being washed away by irrigation or falling out due to gravity. In some situations, the composite product can be packed into irregular voids and can hold a molded shape.

In some instances, the prepared product can be provided in pouches containing an amount of the composite material. For example, a pouch may contain 5 cc or 10 cc of the composite product. As described above, the packaged product may be substantially free of excess cryoprotectant due to the rinsing and/or draining steps.

Adipose Derived Filler Systems and Methods

Embodiments of the present invention encompass decellularized adipose fibrous filler or matrix compositions, and methods for their use and manufacture. For example, adipose derived fibrous filler or matrix materials may be used in reconstructive surgery procedures.

Oftentimes, tissue is removed from a patient during the course of a surgical procedure. In some instances, following surgery, healing of the removal site may result in an indentation or depression of the patient's body. Adipose derived fibrous filler materials as disclosed herein can be used to fill a void where surgical removal of tissue leaves a space that is not natural to the physiology of the removal site. For example, adipose derived fibrous filler materials can be used to aid in the reconstruction of a surgical site where a lumpectomy has been performed. In some instances, adipose derived fibrous filler materials can be used as a space holder to separate two distinct surgical sites. In some instances, adipose derived fibrous filler materials can be used as a matrix that can be combined with other space filling entities.

Upon implantation at a surgical site within a patient, the adipose derived fibrous filler material can operate to fill the site (e.g. lumpectomy location) and maintain its volume, while the site heals. The fibrous filler composition can provide a scaffold that does not change in volume significantly following the time of implant, so as to reconstruct the area, while showing no physical appearance at the site (e.g. little or no indentation following a lumpectomy).

Without being bound by any particular theory, it is believed that by processing the fibers in the manner described herein, the resulting matrix can provide a permanent structure, and structural cavities provided by the structure can become filled with the patient's own fat cells following implantation.

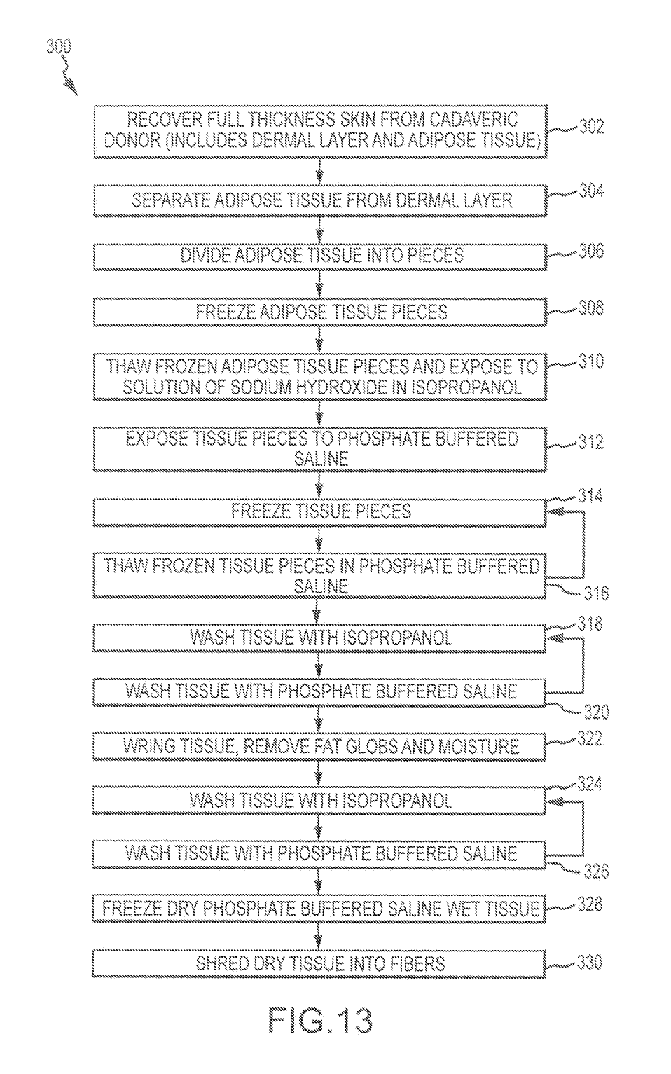

Turning now to the drawings, FIG. 13 shows aspects of an exemplary manufacturing process 300 according to embodiments of the present invention. Exemplary treatment protocols can provide an adipose fibrous structure, for example resembling a non-woven web or fibrous patch, wherein the native microstructure of the adipose ECM is disrupted, and the native macrostructure of the adipose ECM partially retained. Hence, the fibers may not be present in such a way as to resemble a naturally occurring extracellular matrix which has cell related internal structures or architecture such as vascular passages or ductal features. As depicted here at step 302, adipose can be recovered from cadaveric full thickness skin having an ECM, for example following removal of a dermal layer as depicted at step 304. In some cases, the tissue is obtained from the donor's back, abdomen, or thigh area. The adipose can be first sliced into 2-8 mm thick slabs and then cut into 10×10 cm square pieces as depicted at step 306. The pieces can then be frozen in individual packs until needed or desired as depicted at step 308. The tissue can be thawed and exposed to a solution of sodium hydroxide in IPA for 15-45 minutes as depicted at step 310. This solution can cause a small, but not complete disruption in the ECM structure. In some cases, the solution can cause a disruption in cell membranes of the processed tissue. Next the tissue can be exposed to PBS for minutes for pH adjustment as depicted at step 312. The tissue can then be frozen (e.g. by rapid liquid nitrogen freezing) as depicted at 314, and subsequently brought back to room temperature in PBS as depicted at 316. This can be repeated one or more times. Such freezing techniques can operate to cause a separation of oils and water due to freezing point differences. Upon thawing, the tissue may have a leathery appearance, with a reduced oil content. The tissue can then be washed with IPA as depicted at 318 and subsequently with PBS as depicted at 320, and the washes can be repeated as needed or desired. According to some embodiments, one or more freeze/thaw cycles (e.g. dipping in liquid nitrogen) may be sufficient to promote the separation of adequate amounts of oil and water from the adipose tissue ECM material. According to some embodiments, the tissue can be wringed to remove fat globs and moisture as depicted at 322. The tissue can be again washed with IPA as depicted at 324 and then washed with PBS as depicted at 326. The final PBS wet tissue can then be freeze dried as depicted at 328. Once the tissue is dry it can be mechanically shredded into fibers and packaged as depicted at 330. In this way, a packaged adipose derived fibrous filler composition or material can be produced. Fibers can be felt-like and have a pressed web structure. Fibers may also have a non-woven web appearance. In some cases, the fibers are present as a fibrous patch. Often, the fibers are not present in such a way as to resemble a naturally occurring extracellular matrix which has cell related internal structures or architecture such as vascular passages or ductal features. In some embodiments, the fibers are 1-2 cm long and can be pulled apart. In some embodiments, the fibers can be present a residual oil content. Often, the fibers are composed of collagen. Adipose derived fibrous filler compositions can be evaluated by histological methods to determine cell contents (or lack thereof) and general oil content. Relatedly, adipose derived fibrous filler compositions can be evaluated by a variety of techniques to determine their cell content, sterility, biocompatibility, and effectiveness for maintaining an occupied space upon implantation or administration.

In use, adipose derived fibrous filler compositions can be provided to a patient treatment site, thus alleviating the need to subject the patient to secondary surgical procedures to harvest fat tissue for the reconstructive surgery.

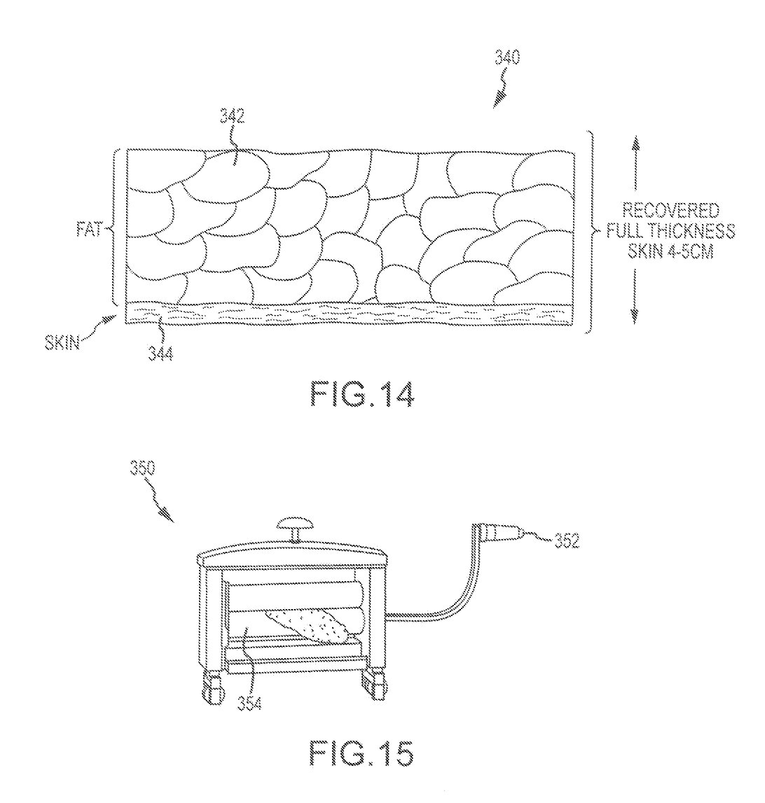

FIG. 14 provides an illustrative example of a starting material for use in preparing an adipose derived fibrous filler material, according to embodiments of the present invention. As depicted here, a portion of full thickness skin 340 can be recovered from a donor. The portion of full thickness skin 340 can have a thickness of about 4 to 5 cm, for example, and can include both fat component 342 and skin component 344.

Processing of the portion of full thickness skin 340 can result in a removed portion of dermis, and a remaining portion or slab of fat (for example having a thickness within a range from about 1 cm to about 5 cm). A matrix can be isolated from the fat portion, for use as a filler in reconstructive surgery. For example, in some cases processing techniques may involve isolating a sheet of fat fibers from the fat portion. In some cases, a dermis decellularization technique that includes 0.1 N NaOH and IPA washings can be used to obtain a fibrous structure from the fat portion.

According to some embodiments, fat slabs can be sliced into individual slabs having a thickness, for example between about 1 cm and about 2 cm, or less. The slabs can be placed in a 10″×10″ stainless steel pan, on a horizontal rotator. The slabs can be exposed to 0.1 N NaOH and IPA (100%) for 30 minutes. The solution can then be changed to PBS. It has been observed that both solutions can operate to extract debris and oils. Subsequently, the slab can be frozen with liquid nitrogen (LN₂) and then thawed out in room temperature PBS. The resulting fibrous slab appeared to have globs trapped in the matrix. The slab was then rotated gently in a solution of 100% IPA for 30 to 45 minutes at room temperature. This step was observed to extract more oils and debris, with the globs persisting within the matrix. As shown in FIG. 15, a wringing device 350, such as a laundry hand wringer, was used to wring the tissue, causing the fat globs to be forced out of the matrix. As shown here, a handle 352 of the wringer device 350 can be used to rotate rollers 354, thus drawing and compressing the slab between the rollers 354, so as to separate the globs from the matrix. The resulting flat matrix was observed to have a fibrous quality. The squeezed fat fibrous tissue was then freeze dried overnight. Subsequently, the tissue was removed from the freeze dryer, and the resultant slabs were observed to have a leather like and fibrous quality or appearance. Histologic evaluations can be performed using hematoxylin-eosin (H&E) and/or uroplakin (URO) staining techniques to determine cell content removal and/or oil content removal.

All patents, patent publications, patent applications, journal articles, books, technical references, and the like discussed in the instant disclosure are incorporated herein by reference in their entirety for all purposes.

It is to be understood that the figures and descriptions of the invention have been simplified to illustrate elements that are relevant for a clear understanding of the invention. It should be appreciated that the figures are presented for illustrative purposes and not as construction drawings. Omitted details and modifications or alternative embodiments are within the purview of persons of ordinary skill in the art.

It can be appreciated that, in certain aspects of the invention, a single component may be replaced by multiple components, and multiple components may be replaced by a single component, to provide an element or structure or to perform a given function or functions. Except where such substitution would not be operative to practice certain embodiments of the invention, such substitution is considered within the scope of the invention.

The examples presented herein are intended to illustrate potential and specific implementations of the invention. It can be appreciated that the examples are intended primarily for purposes of illustration of the invention for those skilled in the art. There may be variations to these diagrams or the operations described herein without departing from the spirit of the invention. For instance, in certain cases, method steps or operations may be performed or executed in differing order, or operations may be added, deleted or modified.

Different arrangements of the components depicted in the drawings or described above, as well as components and steps not shown or described are possible. Similarly, some features and sub-combinations are useful and may be employed without reference to other features and sub-combinations. Embodiments of the invention have been described for illustrative and not restrictive purposes, and alternative embodiments will become apparent to readers of this patent. Accordingly, the present invention is not limited to the embodiments described above or depicted in the drawings, and various embodiments and modifications can be made without departing from the scope of the claims below.

While exemplary embodiments have been described in some detail, by way of example and for clarity of understanding, those of skill in the art will recognize that a variety of modification, adaptations, and changes may be employed. Hence, the scope of the present invention should be limited solely by the claims.

What is claimed is:

1. A method of manufacturing an allogeneic adipose derived carrier for implantation into a patient, comprising:
decellularizing an amount of adipose tissue;
separating a stromal vascular fraction (SVF) from the decellularized adipose tissue;
extracting an organic phase from the decellularized adipose tissue following the SVF separation.

2. The method according to claim 1, comprising combining the organic phase with a granular tissue material.

3. The method according to claim 2, wherein the granular tissue material comprises bone particles.

4. The method according to claim 2, wherein the granular tissue material comprises a member selected from the group consisting of a cortical bone particle and a cancellous bone particle.

5. The method according to claim 2, wherein the granular tissue material comprises bone particles and stem cells.

6. The method according to claim 1, wherein the decellularizing step comprises treating the amount of adipose tissue with collagenase.

7. The method according to claim 1, wherein the organic phase extraction step comprises treating the decellularized adipose tissue with a base solution.

8. The method according to claim 6, wherein the base solution comprises sodium hydroxide.

9. The method according to claim 1, comprising combining the organic phase with a granular tissue material, wherein the amount of adipose tissue and the granular tissue material are from a common donor individual.

10. An allogeneic adipose derived carrier for implantation into a patient, comprising:
an organic phase of decellularized adipose tissue that is substantially free of a stromal vascular fraction.

11. The allogeneic adipose derived carrier according to claim 10, further comprising a granular tissue material.

12. The allogeneic adipose derived carrier according to claim 11, wherein the granular tissue material comprises bone particles.

13. The allogeneic adipose derived carrier according to claim 11, wherein the granular tissue material comprises a member selected from the group consisting of a cortical bone particle and a cancellous bone particle.

14. The allogeneic adipose derived carrier according to claim 11, wherein the granular tissue material comprises bone particles and stem cells.

15. The allogeneic adipose derived carrier according to claim 11, wherein the organic phase of decellularized adipose material and the granular tissue material are from a common donor individual.

16. A method of delivering a granular tissue material to a patient, comprising:
administering the granular tissue material combined with a carrier to a treatment site of the patient,
wherein the carrier comprises an organic phase of decellularized adipose tissue that is substantially free of a stromal vascular fraction.

17. The method according to claim 16, wherein the granular tissue material comprises bone particles.

18. The method according to claim 16, wherein the granular tissue material comprises a member selected from the group consisting of a cortical bone particle and a cancellous bone particle.

19. The method according to claim 16, wherein the granular tissue material comprises bone particles and stem cells.

20. The method according to claim 16, wherein the organic phase of decellularized adipose tissue and the granular tissue material are from a common donor individual.

21. A method of manufacturing an allogeneic adipose derived matrix for implantation into a patient, comprising:
decellularizing an amount of adipose tissue;
separating a stromal vascular fraction (SVF) from the decellularized adipose tissue;
extracting an organic phase from the decellularized adipose tissue following the SVF separation; and
processing the organic phase to provide the adipose derived matrix.

22. The method according to claim 21, comprising combining the matrix with cells, proteins, and/or other large molecules.

23. The method according to claim 21, wherein the decellularizing step comprises treating the amount of adipose tissue with collagenase.

24. The method according to claim 21, wherein the organic phase extraction step comprises treating the decellularized adipose tissue with a base solution.

25. The method according to claim 24, wherein the base solution comprises sodium hydroxide.

26. An allogeneic adipose derived matrix for implantation into a patient, comprising:
a processed organic phase of decellularized adipose tissue that is substantially free of a stromal vascular fraction.

27. The allogeneic adipose derived matrix according to claim 26, further comprising cells, proteins, and/or other large molecules.

28. The allogeneic adipose derived matrix according to claim 27, wherein the processed organic phase of decellularized adipose material and the cells, proteins, and/or other large molecules are from a common donor individual.

29. A method of delivering a treatment material to a patient, comprising:
administering the treatment material combined with a matrix to a treatment site of the patient,
wherein the matrix comprises a processed organic phase of decellularized adipose tissue that is substantially free of a stromal vascular fraction.

30. The method according to claim 29, wherein the treatment material comprises mesenchymal stem cells or platelet-rich plasma.

31. The method according to claim 29, wherein the treatment material comprises cells, proteins, and/or other large molecules.

32. The method according to claim 29, wherein the processed organic phase of decellularized adipose tissue and the treatment material are from a common donor individual.

33. A composite allograft material prepared from tissue obtained from an individual human donor, comprising:
a stem cell component;
a bone component; and
an adipose component.

34. A composition for treating a treatment site in a patient, comprising:
a decellularized adipose fibrous filler material.
